(19) United States
(12) Patent Application Publication
(10) Pub. No.: US 2020/0306213 A1
(43) Pub. Date: Oct. 1, 2020

(54) COMPOUNDS FOR USE IN THE TREATMENT OR PREVENTION OF LOWE SYNDROME OR DENT DISEASE AND METHODS THEREFOR

(71) Applicants: Université de Montréal, Montréal (CA); The University of Manchester, Manchester, Greater Manchester (GB); Centre National De La Recherche Scientifique, Paris, Cedex 16 (FR); Sorbonne Université, Paris, Cedex 05 (FR); Institut Pasteur, Paris, Cedex 15 (FR)

(72) Inventors: Sébastien CARRENO, Montréal (CA); Khaled Ben El Kadhi, Montréal (CA); Arnaud Echard, Paris (FR); Clothilde Cauvin, Arcueil (FR); Martin Lowe, Cheadle Hulme, Cheshire (GB)

(73) Assignees: Université de Montréal, Montréal, QC (CA); The University of Manchester, Manchester, Greater Manchester (GB); Centre National De La Recherche Scientifique, Paris, Cedex 16 (FR); Sorbonne Université, Paris, Cedex 05 (FR); Institut Pasteur, Paris, Cedex 15 (FR)

(21) Appl. No.: 16/650,447

(22) PCT Filed: Sep. 27, 2018
(86) PCT No.: PCT/CA2018/051218

§ 371 Date: Mar. 25, 2020

(30) Foreign Application Priority Data
Sep. 27, 2017 (CA) 2,980,431

(51) Int. Cl. A61K 31/18; A61P 13/12; A61P 27/12; A61P 21/00; A61P 25/28

(52) U.S. Cl. CPC A61K 31/18; A61P 13/12; A61P 25/28; A61P 21/00; A61P 27/12

(57) ABSTRACT

Compounds, compositions and their use in the treatment or prevention of symptoms of Lowe Syndrome and Dent disease 2 are described, for instance, the use of such compounds in the treatment of Lowe Syndrome and Dent disease 2, or in the treatment or prevention of at least one symptom associated therewith. The compounds disclosed are of Formula I:

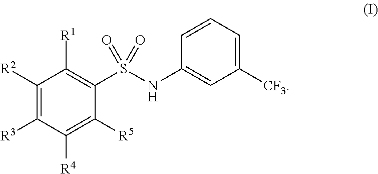

### RELATED APPLICATION

This application claims priority under applicable laws and regulations to Canadian application No. 2,980,431 filed on Sep. 27, 2017, the content of which is incorporated herein by reference in its entirety for all purposes.

### TECHNICAL FIELD

This disclosure generally relates to the treatment of the Lowe syndrome (LS) and/or Dent disease 2 (D2) and to the treatment or prevention of one or more symptoms associated with LS and D2. Disclosed are also compositions for the treatment of LS or D2 and/or symptoms associated therewith.

### BACKGROUND

Lowe syndrome (also called oculocerebrorenal syndrome) is a rare genetic disease affecting approximately 1 in 500,000 births. This X-linked recessive disease develops mostly in men, but some cases affecting women have also been described. This multi-systemic syndrome affects the eyes, the central nervous system and the kidneys. People with Lowe Syndrome suffer from congenital cataracts, glaucomas, hypotonia, mental retardation, aminoaciduria, phosphaturia and low-molecular-weight proteinuria. Dent disease 2 is a type of Dent disease in which patients have the manifestations of Dent disease type 1 (proximal tubule dysfunction and low-molecular-weight proteinuria, associated with hypercalciuria, nephrolithiasis, nephrocalcinosis, and progressive renal failure) associated with extra-renal features. Prevalence of Dent disease of types 1 and 2 is unknown but fewer than 250 families have been reported.

LS and D2 are caused by mutation in the gene encoding the Inositol polyphosphate-5-phosphatase OCRL1. Depletion or inactivation of OCRL1 impairs homeostasis of the PI(4,5)P₂ phosphoinositide, organization of endosomes and cytokinesis. Phosphoinositides (PIPs) are components of cell membranes that regulate various functions. PIPs influence many processes including vesicular trafficking, cell migration and cell division. The amounts and localization of each PIP are regulated by specific kinases, phosphatases and phospholipases. This creates specialized sub-membrane domains with distinct biological functions. Mutations of OCRL1 is at the basis of the molecular dysfunctions causing LS (OMIM #309000) and D2 (OMIM #300555).

The OCRL1 enzyme is an inositol 5-phosphatase that mainly hydrolyses PI(4,5)P₂ into PI(4)P. It was shown to be mainly localized on endomembranes such as the Golgi apparatus and the endosomal system. Under certain circumstances OCRL1 also localizes at the plasma membrane.

The PI(4,5) P2 homeostasis defects observed when OCRL1 is mutated affect cell motility, vesicular trafficking, endocytosis and primary cilia formation (see Coon, B. G. et al., 2009, Hum. Mol. Genet., 18, 4478; Choudhury, R. et al., 2005, Molec. Biol. Of the Cell, 16, 3467; Erdmann, K. S. et al., 2007, Dev. Cell, 13, 377; Luo, N. et al., 2012, Hum. Mol. Genet., 21, 3333; and Coon, B. G. et al., 2012, Hum. Mol. Genet., 21, 1835). Recent work showed that an important function of OCRL1 is to restrict PI(4,5)P₂ at the plasma membrane during cell division, by dephosphorylating this lipid on endomembranes and at specific domains of the plasma membrane (see Ben El Kadhi, K. et al., 2011, Current Biology, 21, 1074; and Dambournet, D. et al., 2011, Nat. Cell Biol., 13, 981). OCRL1 was found to play a key role during cytokinesis, which leads to the physical separation of daughter cells at the end of mitosis. It was also reported a similar requirement of OCRL1 in phagocytosis (Marion, S., et al., 2012, Dev. Cell, 23, 954).

The quality of life of patients suffering from LS is often dramatically affected and depends on the extent of mental and renal manifestations. Symptoms vary widely, since some children are mildly affected and able to attend normal schools with special care, while others are severely affected with loss of vision and mobility. Life expectancy is approximately 30-40 years with death usually occurring between the end of the second decade and the beginning of the fourth (Loi, M., 2006, Orphanet Journal of Rare Diseases, 1, 16, pp. 1-5).

The specific symptoms and severity of Dent disease can vary greatly, even among members of the same family. Signs of Dent disease usually appear in childhood and worsen over time. Common signs of Dent disease include proteinuria, hypercalciuria, nephrocalcinosis, kidney stones that may cause abdominal pain and hematuria. Less commonly, people with Dent disease develop rickets, a bone disorder due to low levels of vitamin D and certain minerals in the blood.

Rickets can be associated with weakening of the bones, bone pain, bowed legs, and difficulty walking. Males with Dent disease 2 are also at increased risk for mild intellectual disability and hypotonia.

Unfortunately, there is no cure currently available for treating Lowe syndrome and Dent disease 2. As such, currently available therapies are limited to the treatment of the clinical manifestations of LS and D2. For instance, people with Lowe syndrome are born with cataracts which are surgically removed during the first weeks of life. Glaucoma develop in half of Lowe syndrome patients and surgery is necessary to restore adequate eye pressure. Most people suffering from Lowe syndrome develop kidney problems during the first year, which aggravates later during life. Medications are necessary to counterbalance the renal losses of electrolytes and other substances. Finally, hypotonia and mental retardation require physical, speech, and feeding therapies.

People suffering from LS and D2 are thus in desperate need of a therapeutic strategy that will target the molecular origin as well as the cellular consequences of the disease.

### SUMMARY

According to one aspect, the present technology relates to a compound for use in the treatment of Lowe Syndrome or Dent disease 2, wherein said compound is of Formula I:

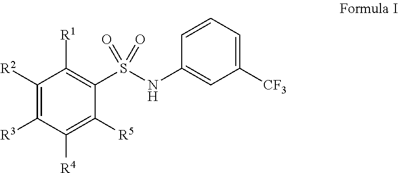

wherein:
R¹ to R⁵ are each independently selected from H, hydroxyl, halogen, C₁₋₄alkyl, —CF₃, —CO₂H, —CO₂R⁶, —OC(O)R⁶, and —(CH₂)ₘCO₂R⁶;
R⁶ is independently in each occurrence a C₁₋₂₀alkyl group; and
m is an integer selected from 1 to 6;
or a pharmaceutically acceptable salt or solvate thereof.

In one embodiment, the compound is 2,4,6-trimethyl-N-(m-3-trifluoromethylphenyl) benzenesulfonamide (m-3M3FBS) or a solvate thereof.

In one embodiment, the compound is for the treatment of Lowe syndrome. In another embodiment, the compound is for the treatment of Dent disease 2.

According to another embodiment, the present technology relates to a compound for use in the treatment or prevention of renal dysfunction associated with Lowe syndrome or Dent disease 2, wherein said compound is of Formula I as herein defined, or a pharmaceutically acceptable salt or solvate thereof. In one embodiment, the compound is 2,4,6-trimethyl-N-(m-3-trifluoromethylphenyl) benzene sulfonamide or a solvate thereof.

In a further embodiment, the treatment comprises restoration of normal cytokinesis or prevention of cytokinetic failure in OCRL1 depleted cells. In another embodiment, the treatment comprises restoration of renal tubule endocytosis.

In a further embodiment, the present description relates to a compound for use in the treatment or prevention of at least one symptom associated with Lowe syndrome or Dent disease 2, wherein said compound is of Formula I as herein defined, or a pharmaceutically acceptable salt or solvate thereof. In one embodiment, the compound is 2,4,6-trimethyl-N-(m-3-trifluoromethylphenyl) benzene sulfonamide or a solvate thereof.

According to one embodiment, the symptom associated with Lowe syndrome includes at least one of brain development damages, congenital cataracts, weak muscle tone, and life-threatening kidney abnormalities. In another embodiment, the symptom associated with Lowe syndrome includes at least one of cataracts, glaucoma, hypotonia, mental retardation, aminoaciduria, phosphaturia and low-molecular-weight proteinuria.

According to another embodiment, the symptom associated with Dent disease 2 comprises at least one of proteinuria, hypercalciuria, nephrolithiasis, nephrocalcinosis, kidney stones that may cause abdominal pain and hematuria, and progressive renal failure. In a further embodiment, the symptom associated with Dent disease 2 comprises at least one of rickets, weakening of the bones, bone pain, bowed legs, difficulty walking, mild intellectual disability and hypotonia.

According to another aspect, the present technology relates to the use of a compound of Formula I as herein defined or a pharmaceutically acceptable salt or solvate thereof, for the treatment of Lowe Syndrome or Dent disease 2. For instance, the compound of Formula I may be 2,4,6-trimethyl-N-(m-3-trifluoromethylphenyl) benzene sulfonamide or a solvate thereof. In another embodiment, the compound is for the treatment of Lowe syndrome. In a further embodiment, the compound is for the treatment of Dent disease 2. In another embodiment, the present relates to the use of a compound of Formula I as herein defined or a pharmaceutically acceptable salt or solvate thereof, for the treatment or prevention of renal dysfunction associated with Lowe syndrome or Dent disease 2. In one embodiment, the compound is 2,4,6-trimethyl-N-(m-3-trifluoromethylphenyl) benzene sulfonamide or a solvate thereof. In a further embodiment, the treatment comprises restoration of normal cytokinesis or prevention of cytokinetic failure in OCRL1 depleted cells. In yet another embodiment, the treatment comprises restoration of renal tubule endocytosis.

In another embodiment, the present document relates to the use of a compound of Formula I as herein defined, or a pharmaceutically acceptable salt or solvate thereof, for the treatment or prevention of at least one symptom associated with Lowe syndrome or Dent disease 2. In one embodiment, the compound is 2,4,6-trimethyl-N-(m-3-trifluoromethylphenyl)benzene sulfonamide or a solvate thereof. According to another embodiment, the symptom associated with Lowe syndrome comprises at least one of brain development damages, congenital cataracts, weak muscle tone, and life-threatening kidney abnormalities. In a further embodiment, the symptom associated with Lowe syndrome comprises at least one of cataracts, glaucoma, hypotonia, mental retardation, aminoaciduria, phosphaturia and low-molecular-weight proteinuria. In yet another embodiment, the symptom associated with Dent disease 2 comprises at least one of proteinuria, hypercalciuria, nephrolithiasis, nephrocalcinosis, kidney stones that may cause abdominal pain and hematuria, and progressive renal failure. In a further embodiment, the symptom associated with Dent disease 2 comprises at least one of rickets, weakening of the bones, bone pain, bowed legs, difficulty walking, mild intellectual disability and hypotonia.

According to another aspect, the present disclosure relates to a method for treating Lowe Syndrome or Dent disease 2, said method comprising administering to a subject in need thereof a compound of Formula I as defined herein, or a pharmaceutically acceptable salt or solvate thereof. For instance, the compound is 2,4,6-trimethyl-N-(m-3-trifluoromethylphenyl) benzenesulfonamide or a solvate thereof. In one embodiment, the method is for treating Lowe syndrome. In another embodiment, the method is for treating Dent disease 2. In another embodiment, the technology described relates to a method for treating or preventing renal dysfunction associated with Lowe syndrome or Dent disease 2, said method comprising administering to a subject in need thereof, a compound of Formula I as defined herein, or a pharmaceutically acceptable salt or solvate thereof (e.g. 2,4,6-trimethyl-N-(m-3-trifluoromethylphenyl)benzene sulfonamide or a solvate thereof). In one embodiment, the treatment comprises restoration of normal cytokinesis or prevention of cytokinetic failure in OCRL1 depleted cells. In another embodiment, the treatment comprises restoration of renal tubule endocytosis.

In another aspect, the present document relates to a method for the treatment or prevention of at least one symptom associated with Lowe syndrome or Dent disease 2, said method comprising administering to a subject in need thereof, a compound of Formula I, or a pharmaceutically acceptable salt or solvate thereof, e.g. 2,4,6-trimethyl-N-(m-3-trifluoromethylphenyl)benzene sulfonamide or a solvate thereof. In one embodiment, the symptom associated with Lowe syndrome comprises at least one of brain development damages, congenital cataracts, weak muscle tone, and life-threatening kidney abnormalities. In another embodiment, the symptom associated with Lowe syndrome comprises at least one of cataracts, glaucoma, hypotonia, mental retardation, aminoaciduria, phosphaturia and low-molecular-weight proteinuria. In a further embodiment, the symptom associated with Dent disease 2 comprises at least one of proteinuria, hypercalciuria, nephrolithiasis, nephrocalcinosis, kidney stones that may cause abdominal pain and hematuria, and progressive renal failure. In yet another embodiment, the symptom associated with Dent disease 2 comprises at least one of rickets, weakening of the bones, bone pain, bowed legs, difficulty walking, mild intellectual disability and hypotonia.

According to a further aspect, the present technology relates to a method for the restoration of normal cytokinesis or prevention of cytokinetic failure in OCRL1 depleted cells, the method comprising contacting said cells with a compound of Formula I or a pharmaceutically acceptable salt or solvate thereof, e.g. 2,4,6-trimethyl-N-(m-3-trifluoromethylphenyl)benzene sulfonamide or a solvate thereof.

According to yet another aspect, described is a method for the prevention of cytokinesis failure in dOCRL depleted cells, the method comprising contacting said cells with a compound of Formula I, or a pharmaceutically acceptable salt or solvate thereof, e.g. 2,4,6-trimethyl-N-(m-3-trifluoromethylphenyl)benzene sulfonamide or a solvate thereof.

According to a further aspect, the present technology relates to a pharmaceutical composition for a use as defined in any one of the aforementioned embodiments, the composition comprising a compound as herein defined together with a pharmaceutically acceptable carrier, diluent or excipient.

Additional objects and features of the present compound, compositions, methods and uses will become more apparent upon reading of the following non-restrictive description of exemplary embodiments, which should not be interpreted as limiting the scope of the invention.

### BRIEF DESCRIPTION OF THE DRAWINGS

FIG. 1 displays results obtained for (a) control drosophila S2 cells treated with m-3M3FBS, and for dOCRL dsRNA drosophila S2 cells treated with (b) o-3M3FBS or (c) m-3M3FBS.

FIG. 2 is a histogram representing the percentage of multinucleated drosophila S2 cells after various treatments.

FIG. 3 shows the distribution of abscission times in the indicated normal or Lowe syndrome patient renal cell populations after treatment with o-3M3FBS or m-3M3FBS.

FIG. 4 is a histogram illustrating the mean abscission times measured on time lapse movies in the indicated normal or Lowe syndrome patient renal cell populations after treatment with o-3M3FBS or m-3M3FBS.

FIG. 5 shows the cumulative distribution curves of abscission times in OCRL1 depleted HeLa cells in the presence of o-3M3FBS or m-3M3FBS.

FIG. 6 is a histogram illustrating the mean abscission times measured on time lapse movies in control-depleted and in OCRL1-depleted HeLa cells.

FIG. 7 shows the confocal images of pronephric tubules (indicated by dashed lines) in wildtype (WT) ((a) control, (b) o-3M3FBS treated, and (c) m-3M3FBS treated) or OCRL−/− mutant ((d) control, (e) o-3M3FBS treated, and (f) m-3M3FBS treated) zebrafish embryos.

FIG. 8 is a histogram representing the pronephric uptake of a tracer dye in wildtype (WT) or OCRL−/− mutant zebrafish embryos monitored by fluorescence microscopy.

### DETAILED DESCRIPTION

All technical and scientific terms and expressions used herein have the same definitions as those commonly understood by a person skilled in the art to which the present technology pertains. The definition of some terms and expressions used is nevertheless provided below. To the extent the definitions of terms in the publications, patents, and patent applications incorporated herein by reference are contrary to the definitions set forth in this specification, the definitions in this specification will control. The section headings used herein are for organizational purposes only, and are not to be construed as limiting the subject matter disclosed.

i. Definitions

Chemical structures described herein are drawn according to conventional standards. Also, when an atom, such as a carbon atom, as drawn seems to include an incomplete valency, then the valency is assumed to be satisfied by one or more hydrogen atoms even though these are not necessarily explicitly drawn. Hydrogen atoms should be inferred to be part of the compound.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the content clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Unless otherwise stated, structures depicted herein are also meant to include all isomeric (e.g., enantiomeric, diastereomeric, and geometric (or conformational)) forms of the structure when applicable; for example, the R and S configurations for each asymmetric center. Therefore, single stereochemical isomers as well as enantiomeric, diastereomeric, and geometric (or conformational) mixtures of the present compounds are within the scope of the present description. The therapeutic compound unless otherwise noted, also encompasses all possible tautomeric forms of the illustrated compound, if any. The term also includes isotopically labeled compounds where one or more atoms have an atomic mass different from the atomic mass most abundantly found in nature. Examples of isotopes that may be incorporated into the compounds of the present invention include, but are not limited to, ²H (D), ³H (T), ¹¹C, ¹³C, ¹⁴C, ¹⁵N, ¹⁸O, ¹⁷O, any one of the isotopes of sulfur, etc. The compound may also exist in unsolvated forms as well as solvated forms, including hydrated forms. The compound may exist in multiple crystalline or amorphous forms. In general, all physical forms are equivalent for the uses contemplated herein and are intended to be within the scope of the present invention.

The expression “pharmaceutically acceptable salt” refers to those salts of the compounds of the present description which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

The term “solvate” refers to a physical association of one of the present compound with one or more solvent molecules, including water and non-aqueous solvent molecules. This physical association may include hydrogen bonding. In certain instances, the solvate will be capable of isolation, for example when one or more solvent molecules are incorporated in the crystal lattice of a crystalline solid. The term “solvate” encompasses both solution-phase and isolable solvates. Exemplary solvates include, without limitation, hydrates, hemihydrates, ethanolates, hemiethanolates, n-propanolates, iso-propanolates, 1-butanolates, 2-butanolate, and solvates of other physiologically acceptable solvents, such as the Class 3 solvents described in the International Conference on Harmonization (ICH), Guide for Industry, Q3C Impurities: Residual Solvents (1997). Accordingly, the compound as herein described also includes each of its solvates and mixtures thereof.

ii. Phospholipase C Activator

A phospholipase C (PLC) activator is a molecule capable of activating or reactivating a PLC enzyme function. Examples of PLC activators are illustrated by general Formula I:

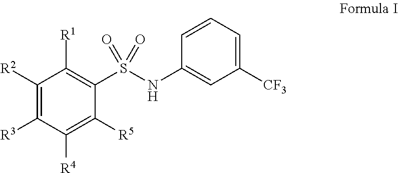

wherein:

R¹ to R⁵ are each independently selected from H, hydroxyl, halogen (e.g. F or Cl), C₁₋₄alkyl, —CF₃, —CO₂H, —CO₂R⁶, —OC(O)R⁶, and —(CH₂)ₘCO₂R⁶;

R⁶ is independently in each occurrence a C₁₋₂₀alkyl group, preferably a C₁₋₄alkyl group; and

m is an integer selected from 1 to 6;

or a pharmaceutically acceptable salt or solvate thereof.

According to another example, the compound is of Formula I, wherein R¹, R³ and R⁵ are each a methyl group and R² and R⁴ are each hydrogen atoms. In another example, the compound is of Formula I, wherein R² and R⁴ are each a t-butyl group, R³ is a fluorine atom, and R¹ and R⁵ are each hydrogen atoms. In yet another example, the compound is of Formula I, wherein R³ is a t-butyl group, and R¹, R², R⁴, and R⁵ are each hydrogen atoms.

Accordingly, an example of a PLC activator is 2,4,6-trimethyl-N-(m-3-trifluoromethylphenyl) benzene sulfonamide (m-3M3FBS), more specifically represented by the following formula:

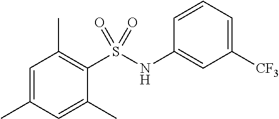 [SMILES: Cc1cc(C)c(S(=O)(=O)Nc2cccc(C(F)(F)F)c2)c(C)c1]

The compound may be in any amorphous, crystalline or polymorphic form, including any solvates, or a mixture thereof.

The compounds described herein may be prepared by any method known to a skilled medicinal chemist or may be commercially available.

iii. Methods, Uses, Formulations and Administration

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in treatment, healing, prevention, or amelioration of a disease, disorder, or symptom thereof, or a decrease in the rate of advancement of a disease or disorder. The term also includes within its scope amounts effective to enhance normal physiological function.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Patients suffering from Lowe syndrome (LS or oculocerebrorenal syndrome) present brain development damages, congenital cataracts, weak muscle tone, life threatening kidney abnormalities. As such, symptoms of LS include cataracts, glaucoma, hypotonia, mental retardation, aminoaciduria, phosphaturia and/or low-molecular-weight proteinuria. Symptoms of Dent disease 2 include proteinuria, hypercalciuria, nephrolithiasis, nephrocalcinosis, kidney stones that may cause abdominal pain and hematuria, and progressive renal failure. Less commonly, people with Dent disease develop rickets, a bone disorder due to low levels of vitamin D and certain minerals in the blood. Rickets can be associated with weakening of the bones, bone pain, bowed legs, and difficulty walking. Males with Dent disease 2 are also at increased risk for mild intellectual disability and hypotonia.

It was discovered that overexpression of PTEN restores PI(4,5)P₂ homeostasis in dOCRL depleted cells by reducing its levels on endomembrane and rescues cytokinesis defects of dOCRL depleted cells. A new signaling network was identified by which PTEN promotes hydrolysis of PI(4,5)P₂ on endomembrane, for instance, by activating an atypical phospholipase C (PLC). As such, activation of PLC rescues the cytokinesis defects observed when OCRL1 is inactivated. Such an activation may be accomplished using a chemical activator. Both LS and D2 are associated with a loss in function of OCRL.

Accordingly, the present disclosure relates to the treatment of LS or D2 in a subject, including the treatment, alleviation, mitigation or prevention of at least one symptom associated with LS or D2 using a PLC activator. As used herein, the term “PLC activator” is defined as a compound that stimulates, activates or reactivates a PLC enzyme or one of its functions with measurable activity, for instance, as defined in section (ii) of the present description.

The term “measurable activity” as used herein, means a measurable change in activity of at least one PLC functions between a sample comprising a provided compound, or composition thereof, and an equivalent sample without said compound, or composition thereof.

The term “patient or subject” as used herein refers to an animal such as a mammal. A subject may therefore refer to, for example, fish, dogs, cats, horses, cows, pigs, guinea pigs, and the like. Preferably the subject is a human.

The present description therefore further relates to a method of treating a subject, such as a human, suffering from at least one symptom associated with Lowe syndrome or Dent Disease 2. The method comprises administering a therapeutically effective amount of a PLC activator, which functions by activating a PLC and rescuing an OCRL1 function, to a subject in need of such treatment.

In certain embodiments, the present description provides a method of treating a disorder (as described herein) in a subject, comprising administering to the subject identified as in need thereof, a compound of the present description. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination, medical/family history, and genetic determination.

A method of assessing the efficacy of a treatment in a subject includes determining the pre-treatment symptoms of a disorder by methods well known in the art and then administering a therapeutically effective amount of a compound of the present description, to the subject. After an appropriate period of time following the administration of the compound (e.g., 1 week, 2 weeks, one month, six months), the symptoms of the disorder are determined again. The modulation (e.g., decrease) of symptoms of the disorder indicates efficacy of the treatment. The symptoms of the disorder may be determined periodically throughout treatment. For example, the symptoms of the disorder may be checked every few days, weeks or months to assess the further efficacy of the treatment. A decrease in symptoms of the disorder indicates that the treatment is efficacious. The method described may also be used to screen or select patients that may benefit from treatment with the present PLC activator.

According to one aspect, there is provided a method for identifying compounds for use in treating Lowe syndrome or Dent disease 2 which comprises the step of determining whether the compound activates a PLC enzyme function and/or rescues a depleted OCRL function.

A method of the present description also comprises the treatment of Lowe syndrome or Dent disease 2, the method comprising administering a compound as herein defined. For example, such treatment comprises restoration of normal cytokinesis or prevention of cytokinetic failure in OCRL1 depleted cells and/or restoration of renal tubule endocytosis.

In another aspect, provided in a method for the prevention or treatment of one or more symptoms or manifestations associated with Lowe syndrome, including one or more of brain development damages, congenital cataracts, weak muscle tone, and life-threatening kidney abnormalities, the method comprising a step of administering a compound or composition as herein described.

In another aspect, provided in a method for the prevention or treatment of one or more symptoms or manifestations associated with Lowe syndrome, including one or more of cataracts, glaucomas, hypotonia, mental retardation, aminoaciduria, phosphaturia and low-molecular-weight proteinuria.

In another aspect, provided in a method for the prevention or treatment of one or more symptoms or manifestations associated with Dent disease 2, including one or more of proteinuria, hypercalciuria, nephrolithiasis, nephrocalcinosis, kidney stones that may cause abdominal pain and hematuria, progressive renal failure. Additional symptoms or manifestations associated Dent disease 2 include rickets, weakening of the bones, bone pain, bowed legs, difficulty walking, mild intellectual disability and hypotonia.

Also contemplated is a method for the treatment or prevention of renal dysfunction in a subject with Lowe syndrome or Dent disease 2, the method comprising administering a compound as herein defined. For instance, the treatment comprises restoration of normal cytokinesis or prevention of cytokinetic failure in OCRL1 depleted cells, and/or restoration of renal tubule endocytosis.

According to another embodiment, the description provides a method of restoring normal cytokinesis or prevention of cytokinetic failure in OCRL1 depleted cells using a composition comprising a compound of the present description and a pharmaceutically acceptable carrier, adjuvant, or vehicle. For instance, the amount of the compound in a provided composition is such that it is effective to measurably restore normal cytokinesis or prevent cytokinetic failure in OCRL1 depleted cells. More specifically, the OCRL1 depleted cells are cells of subject having Lowe Syndrome or Dent disease 2.

In some embodiments, the therapeutically effective amount of a compound as defined herein can be administered to a patient alone or in a composition, admixed with a pharmaceutically acceptable carrier, adjuvant, or vehicle.

The expression “pharmaceutically acceptable carrier, adjuvant, or vehicle” and equivalent expressions, refer to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions described herein may be administered orally, parenterally, by inhalation spray, topically, rectally, nasally, buccally, or via an implanted reservoir. The term “parenteral” as used herein includes subcutaneous, intravenous, intramuscular, intraarticular, intrasynovial, intrasternal, intrathecal, intrahepatic, intralesional and intracranial injection or infusion techniques. Other modes of administration also include intradermal or transdermal administration.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly(anhydrides). Depot injectable formulations are also prepared by entrapping the compound in liposomes or microemulsions that are compatible with body tissues.

Compositions for rectal administration are preferably suppositories which can be prepared by mixing the compounds of the present description with suitable non-irritating excipients or carriers such as cocoa butter, polyethylene glycol or a suppository wax which are solid at ambient temperature but liquid at body temperature and therefore melt in the rectum and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone (PVP), sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

PLC activators can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

Pharmaceutically acceptable compositions provided herein may be formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compound that may be combined with carrier materials to produce a composition in a single dosage form will vary depending upon the patient to be treated and the particular mode of administration. Provided compositions may be formulated such that a dosage of between 0.01-100 mg/kg body weight/day of the activator can be administered to a patient receiving these compositions.

It should also be understood that a specific dosage and treatment regimen for any particular patient will depend upon a variety of factors, including age, body weight, general health, sex, diet, time of administration, rate of excretion, drug combination, the judgment of the treating physician, and the severity of the symptoms associated with Lowe syndrome. The amount of a provided compound in the composition will also depend upon the particular compound in the composition.

Compound or compositions described herein may be administered using any amount and any route of administration effective for treating or lessening the severity of the symptoms as contemplated herein. The exact amount required will vary from subject to subject, depending on the species, age, and general condition of the subject, the severity of the infection, the particular agent, its mode of administration, and the like. Provided compounds are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment.

Pharmaceutically acceptable compositions of this disclosure can be administered to humans and other animals orally, rectally, parenterally, intracisternally, intraperitoneally, topically (as by powders, ointments, or drops), buccally, as an oral or nasal spray, or the like, depending on the severity of the infection being treated. In certain embodiments, provided compounds may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Depending upon the Lowe syndrome symptoms to be treated, additional therapeutic agents may also be present in the compositions of this disclosure or administered separately as part of a dosage regimen.

Upon improvement of a subject's condition, a maintenance dose of a compound or composition of the present description may be administered. Subsequently, the dosage or frequency of administration, or both, may be reduced, as a function of the symptoms, to a level at which the improved condition is retained when the symptoms have been alleviated to the desired level. The subject may require treatment on a long-term basis to prevent recurrence of disease symptoms.

It will be understood, however, that the total daily usage of the compound and compositions of the present description will be decided by the attending physician within the scope of sound medical judgment. The total daily activating dose of the compound of the present description administered to a subject in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In one embodiment, treatment regimens according to the present description comprise administration to a patient in need of such treatment from about 10 mg to about 1000 mg of the compound(s) of the present description per day in single or multiple doses.

The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

The following non-limiting examples are illustrative embodiments and should not be construed as further limiting the scope of the present invention. These examples will be better understood with reference to the accompanying figures.

The Examples set forth herein below provide syntheses and experimental results obtained for certain exemplary compounds. Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

### Example 1

### In Vitro Assay in a Drosophila Cell Model

Drosophila S2 cells were grown in Schneider's Drosophila medium complemented with FBS, Penicillin and Streptomycin (Invitrogen). Control cells or dOCRL dsRNA treated cells were treated for 24 hours with 50 μM of the PLC activator m-3M3FBS or 50 μM of its inactive analog o-3M3FBS. Cells were fixed and labelled for F-Actin (red) and DNA (blue).

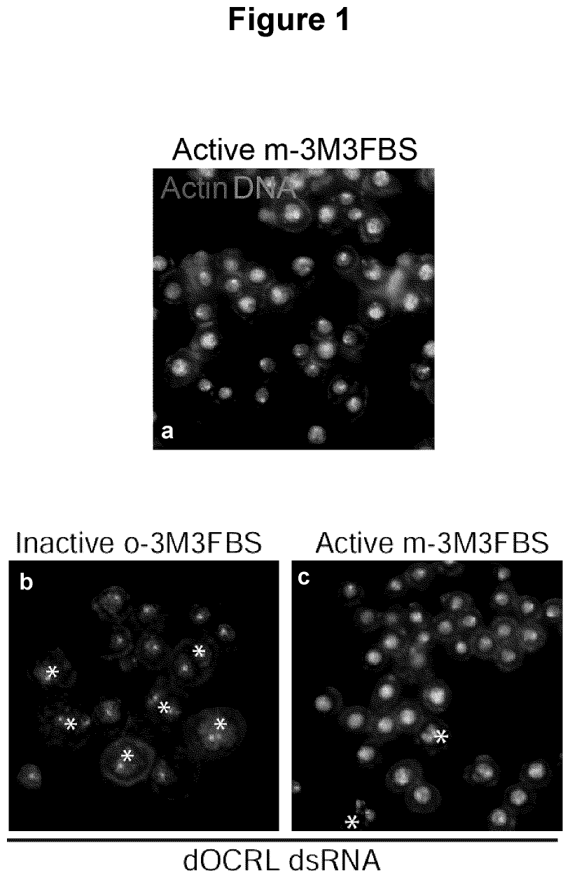

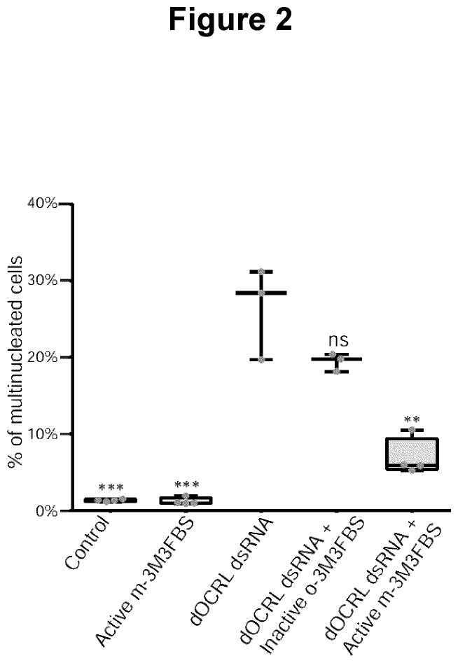

Images are shown in FIGS. 1(a) to 1(c) in which stars mark multinucleated cells. The histogram of FIG. 2 represents the percentage of multinucleated cells quantified following the indicated treatments. Each P-values were calculated against dOCRL dsRNA treated cells.

It was thus found that while m-3M3FBS did not perturb cytokinesis in control cells, it prevented cytokinesis failure in dOCRL depleted cells. o-3M3FBS, its inactive analog, did not significantly modify the multi-nucleation rate of dOCRL depleted cells.

### Example 2

### In Vitro Abscission Assay in Human Cells

Further tests were carried out using human cellular models for the Lowe syndrome. It was previously reported that Hela cells in which OCRL1 was depleted by RNAi or renal epithelial cells of a Lowe syndrome patient harboring a catalytically inactive version of OCRL1 present an important delay in cytokinetic abscission (described in Dambournet, D. et al., 2011, Nat. Cell Biol., 13, 981-8). m-3M3FBS and its inactive isomer o-3M3FBS were tested in both models and compared to control cells.

a) In Vitro Assay in Lowe Syndrome Human Patient Cells

In this experiment, normal renal epithelial cells from a donor not mutated in OCRL1 and renal epithelial cells from a Lowe syndrome patient were grown in DMEM/F12 (Gibco BRL) supplemented with 10% fetal bovine serum, ITS supplemented, 4 pg/mL triiodothyronine, 36 ng/mL dexamethasone, 10 ng/ml EGF, 100 U/mL penicillin/streptomycin and 2 mM glutamine at 33° C. Normal cells and Lowe syndrome cells were treated with the PLC activator m-3M3FBS or its inactive o-3M3FBS analog diluted in DMSO, used each at 25 μM during the full duration (60 hours) of time-lapse recording.

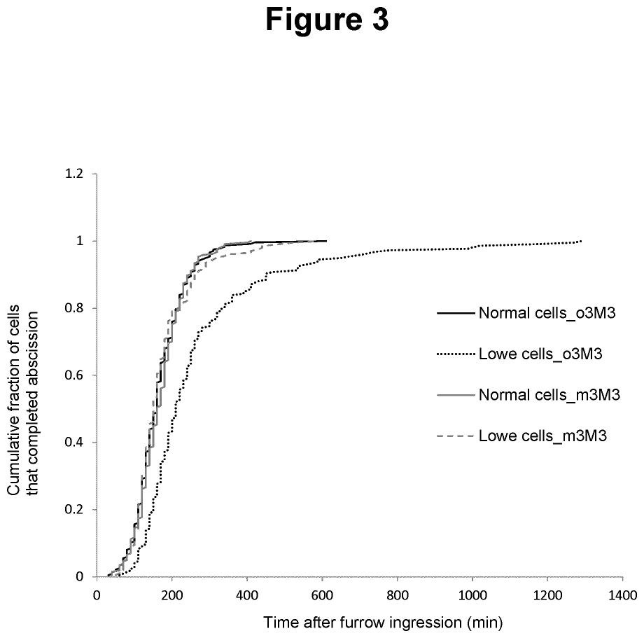

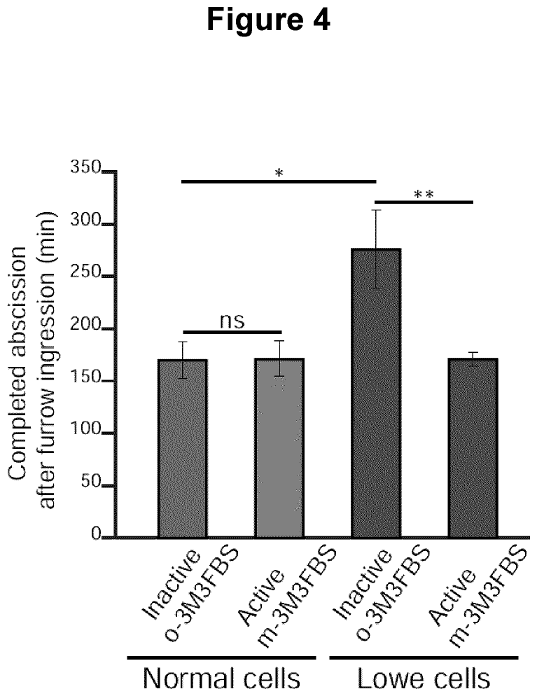

Cell divisions were recorded by time-lapse microscopy using a Nikon Eclipse Ti Inverted Microscope. The curves represent the cumulative distribution of the abscission times in the indicated cell populations (see FIG. 3). Mean abscission times were measured from these time-lapse movies in normal and Lowe renal epithelial cells treated with the PLC activator m-3M3FBS or its inactive analog o-3M3FBS (see FIG. 4). Results show that the abscission delay observed in Lowe patient cells was fully corrected by treatment with m-3M3FBS while the timing of abscission was not affected in normal cells. Comparatively, abscission delay observed in Lowe patient cells was not corrected by treatment with o-3M3FBS.

b) In Vitro Assay in Human HeLa Epithelial Cells

Human HeLa epithelial cells were grown in DMEM medium (Gibco BRL) supplemented with 10% fetal bovine serum, 100 U/ml penicillin/streptomycin and 2 mM glutamine at 37° C. HeLa cells were depleted in OCRL1 by transfecting control siRNAs (5′-CGUACGCGGAAUACUUCGA-3′) or OCRL1 RNAi (5′-GAAAGGAUCAGUGUCGAUA-3′) specific siRNAs for 72 h before imaging using HiPerFect reagents (Qiagen). Depleted cells were treated with the PLC activator m-3M3FBS or its inactive o-3M3FBS analog diluted in DMSO, used each at 25 μM during the full duration (60 hours) of time-lapse recording.

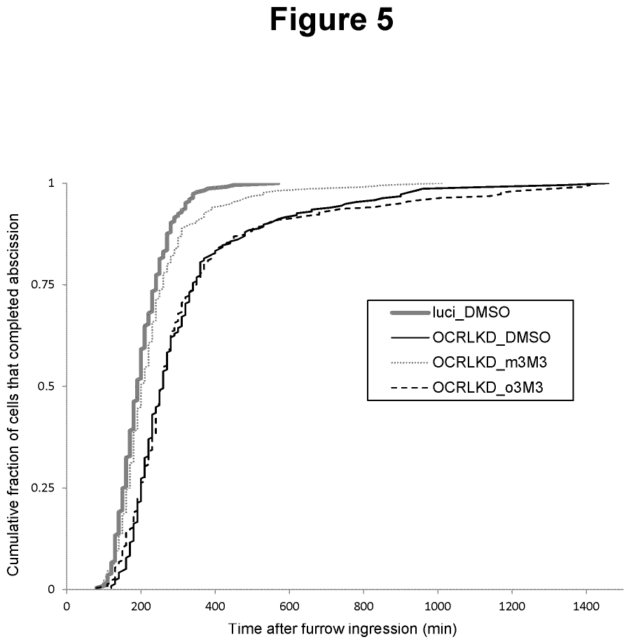

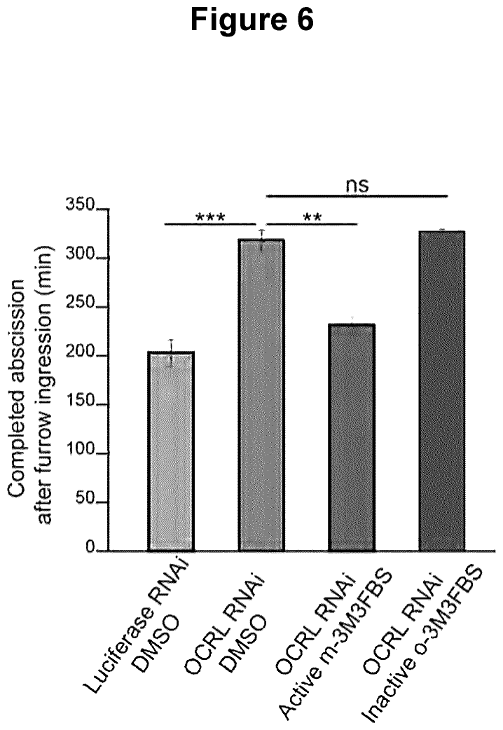

Cell divisions were recorded by time-lapse microscopy using a Nikon Eclipse Ti Inverted Microscope. The curves represent the cumulative distribution of the abscission times in the indicated cell populations (see FIG. 5). Mean abscission times were measured from these time-lapse movies in control-depleted and in OCRL1-depleted cells treated with the PLC activator m-3M3FBS or its inactive analog o-3M3FBS (see FIG. 6). Results of Example 2(a) were confirmed, where the PLC activator m-3M3FBS showed correction of the abscission delay in OCRL1-depleted cells while o-3M3FBS had no significant effect on abscission compared to control (DMSO alone).

Therefore, it can be concluded that the abscission delay resulting from OCRL inactivation or depletion is corrected by low doses of the PLC activator m-3M3FBS.

### Example 3

### In Vivo Assay in a Zebrafish Model

Finally, to test whether activation of PLC rescues phenotypes induced by loss of OCRL1 in an in vivo context, a previously established Ocrl−/− mutant zebrafish model for Lowe syndrome was used (Ramirez I. B. et al., 2012, Hum. Mol. Genet., 21, 1744-59, Oltrabella F., et al., 2015, PLoS Genetics, 11(4):e1005058). Wild type fish were of AB background.

Lysine-fixable 10 kDa dextran labelled with Alexa 488 (Molecular Probes) was prepared in PBS at 2 μg/μl final concentration. Zebrafish embryos at 72 hpf (hours post fertilisation) were treated for 60 minutes with DMSO control (0.005% DMSO), 5 μM m-3M3FBS or 5 μM o-3M3FBS by addition to the water. Embryos were then anaesthetised with 0.2 mg/ml MS222 (Sigma) in chorion water, and the tracer Alexa 488-10 kDa dextran (green) was injected into the common cardinal vein using a glass micropipette PLI-90 Pico-Injector (Harvard Apparatus). Embryos were returned to the respective drug treatments and incubated at 29° C. Pronephric accumulation was assessed 2 hours after injection on whole mount embryos using a fluorescent dissecting stereomicroscope (Leica MZ10F). Statistical analysis was performed using the Pearson's chi-squared test with Prism software (Prism Software Corporation).

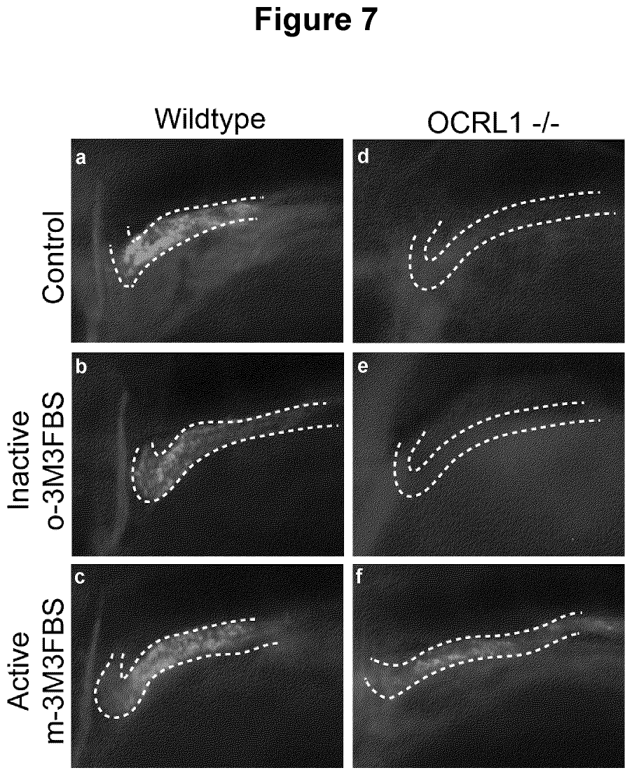

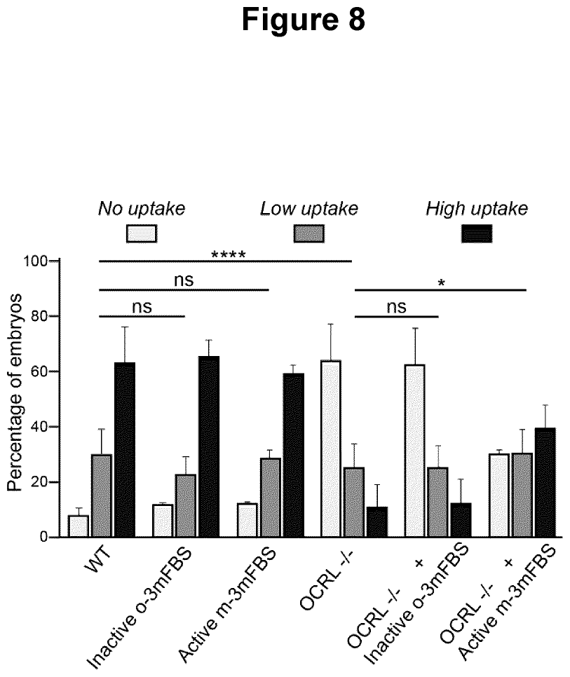

FIG. 7 shows confocal images of pronephric tubules (indicated by a dashed line) in wildtype (FIGS. 7a to 7c) and Ocrl−/− zebrafish mutant embryos (FIGS. 7d to 7f). FIG. 8 shows the pronephric accumulation in the indicated embryos (wildtype (WT) or) as monitored by fluorescence microscopy, ns: non-significant; *, P<0.05; **, P<0.01; ***, P<0.001; ****, P<0.0001 (Student t-test). Bar 10 μm.

As shown in FIGS. 7 and 8, m-3M3FBS treatment had no effect upon renal uptake in wildtype embryos, but efficiently rescued endocytosis in the renal tubule of OCRL1 mutant embryos. The inactive analog had no effect in either zebrafish strains. These results indicate that activation of PLC with m-3M3FBS can rescue loss of OCRL1 function in an in vivo context, namely the renal tubule, one of the major tissues affected in Lowe syndrome patients.

Numerous modifications could be made to any of the embodiments described above without departing from the scope of the present invention. Any references, patents or scientific literature documents referred to in the present document are incorporated herein by reference in their entirety for all purposes.

1-14. (canceled)

15. A method for treating Lowe Syndrome or Dent disease 2, comprising:
administering to a subject in need thereof a compound of Formula I: 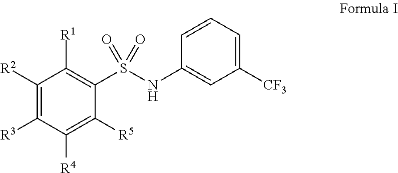
wherein:
R¹ to R⁵ are each independently selected from H, hydroxyl, halogen, C₁₋₄ alkyl, —CF₃, —CO₂H, —CO₂R⁶, —OC(O)R⁶, and —(CH₂)ₘCO₇R⁶;
R⁶ is independently in each occurrence a C₁₋₂₀ alkyl group; and
m is an integer selected from 1 to 6;
or a pharmaceutically acceptable salt or solvate thereof.

16. The method of claim 15, wherein said compound is 2,4,6-trimethyl-N-(m-3-trifluoromethylphenyl)benzene sulfonamide or a solvate thereof.

17. The method of claim 15, for treating Lowe syndrome.

18. The method of claim 15, for treating Dent disease 2.

19. A method for treating or preventing renal dysfunction associated with Lowe syndrome or Dent disease 2. said method comprising:
administering to a subject in need thereof, a compound of Formula I: 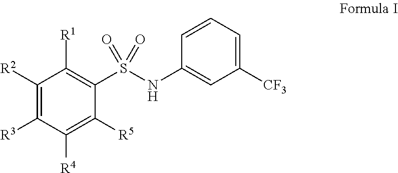
wherein:
R¹ to R⁵ are each independently selected from H, hydroxyl, halogen, C₁₋₄ alkyl, —CF₃, —CO₂H, —CO₂R⁶, —OC₁₋₄ alkyl, —OC(O)R⁶, and —(CH₂)ₘCO₂R⁶;
R⁶ is independently in each occurrence a C₁₋₂₀ alkyl group; and
m is an integer selected from 1 to 6;
or a pharmaceutically acceptable salt or solvate thereof.

20. The method of claim 19, wherein said compound is 2,4,6-trimethyl-N-(m-3-trifluoromethylphenyl)benzene sulfonamide or a solvate thereof.

21. The method of claim 15, wherein said treatment comprises restoring normal cytokinesis or preventing cytokinetic failure in OCRL1 depleted cells in the subject.

22. The method of claim 15, wherein said treatment comprises restoring renal tubule endocytosis in the subject.

23. A method for treatment or prevention of at least one symptom associated with Lowe syndrome or Dent disease 2, comprising:
administering to a subject in need thereof, a compound of Formula I: 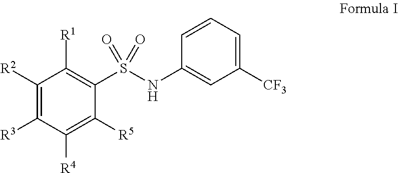
wherein:
R¹ to R⁵ are each independently selected from H, hydroxyl, halogen, C₁₋₄ alkyl, —CF₃, —CO₂H, —CO₂R⁶, —OC₁₋₄ alkyl, —OC(O)R⁶, and —(CH₂)ₘCO₂R⁶;
R⁶ is independently in each occurrence a C₁₋₂₀ alkyl group; and
m is an integer selected from 1 to 6;
or a pharmaceutically acceptable salt or solvate thereof.

24. The method of claim 23, wherein said compound is 2,4,6-trimethyl-N-(m-3-trifluoromethylphenyl)benzene sulfonamide or a solvate thereof.

25. The method of claim 23, wherein said at least one symptom associated with Lowe syndrome is at least one selected from the group consisting of brain development damages, congenital cataracts, weak muscle tone, and life-threatening kidney abnormalities.

26. The method of claim 23, wherein said at least one symptom associated with Lowe syndrome is at least one selected from the group consisting of cataracts, glaucoma, hypotonia, mental retardation, aminoaciduria, phosphaturia, and low-molecular-weight proteinuria.

27. The method of claim 23, wherein said at least one symptom associated with Dent disease 2 is at least one selected from the group consisting of proteinuria, hypercalciuria, nephrolithiasis, nephrocalcinosis, kidney stories that may cause abdominal pain and hematuria, and progressive renal failure.

28. The method of claim 23, wherein said at least one symptom associated with Dent disease 2 is at least one selected from the group consisting of rickets, weakening of the bones, bone pain, bowed legs, difficulty walking, mild intellectual disability, and hypotonia.

29. A method for restoration of normal cytokinesis or prevention of cytokinetic failure in OCRL1 depleted cells, comprising:
contacting said cells with a compound of Formula I: 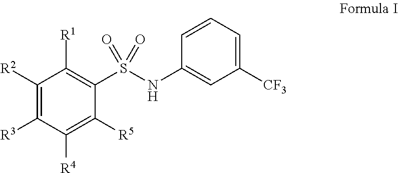
wherein:
R¹ to R⁵ are each independently selected from H, hydroxyl, halogen, C₁₋₄ alkyl, —CF₃, —CO₂H, —CO₂R⁶, —OC₁₋₄ alkyl, —OC(O)R⁶, and —(CH₂)ₘCO₂R⁶;
R⁶ is independently in each occurrence a C₁₋₂₀ alkyl group; and
m is an integer selected from 1 to 6;
or a pharmaceutically acceptable salt or solvate thereof.

30. A method for prevention of cytokinesis failure in dOCRL depleted cells, comprising:
contacting said cells with a compound of Formula I: 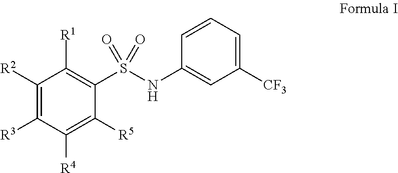
wherein:
R¹ to R⁵ are each independently selected from H, hydroxyl, halogen, C₁₋₄ alkyl, —CF₃, —CO₂H, —CO₂R⁶, —OC₁₋₄ alkyl, and —(CH₂)ₘCO₂R⁶;
R⁶ is independently in each occurrence a C₁₋₂₀ alkyl group; and
in is an integer selected from 1 to 6;
or a pharmaceutically acceptable salt or solvate thereof.

31. The method of claim 29, wherein said compound is 2,4,6-trimethyl-N-(m-3-trifluoromethylphenyl)benzene sulfonamide or a solvate thereof.

32. The method of claim 19, wherein said treatment comprises restoring normal cytokinesis or preventing cytokinetic failure in OCRL1 depleted cells in the subject.

33. The method of claim 19, wherein said treatment comprises restoring renal tubule endocytosis in the subject.

34. The method of claim 30, wherein said compound is 2,4,6-trimethyl-N-(m-3-trifluoromethylphenyl)benzene sulfonamide or a solvate thereof.
